(19) United States
(12) Patent Application Publication
(10) Pub. No.: US 2007/0055173 A1
(43) Pub. Date: Mar. 8, 2007

(54) Rotational core biopsy device with liquid cryogen adhesion probe

(75) Inventors: Russell L. DeLonzor, Pleasanton, CA (US); Richard K. Spero, Pleasanton, CA (US); Christopher D. Owen, Pleasanton, CA (US); Matthew J. Nalipinski, Pleasanton, CA (US); Stephen L. Daleo, Pleasanton, CA (US)

Correspondence Address: CROCKETT & CROCKETT, 24012 CALLE DE LA PLATA, SUITE 400, LAGUNA HILLS, CA, 92653, US

(73) Assignee: Sanarus Medical, Inc.

(21) Appl. No.: 11/210,436

(22) Filed: Aug. 23, 2005

(51) Int. Cl. A61B 10/00; A61B 18/18; A61B 17/32; A61B 17/34

(52) U.S. Cl. 600/564; 606/167; 606/170; 606/184; 606/185; 606/21; 606/22; 606/25; 600/567; 600/568

(57) ABSTRACT

A biopsy device comprising a housing and a releasable coring module having an internal needle adapted for securing a suspect mass to the internal needle and an external cutting cannula adapted to slide over the internal needle to cut the suspect mass from any surrounding tissue.

### FIELD OF THE INVENTIONS

The devices and methods described below relate to the diagnosis and treatment of breast lesions, and more generally, to the diagnosis and treatment of tumors and lesions throughout the body.

### BACKGROUND OF THE INVENTIONS

Biopsy is an important procedure used for the diagnosis of patients with cancerous tumors, pre-malignant conditions, and other diseases and disorders. Typically, in the case of cancer, when the physician establishes by means of procedures such as palpation, mammography or x-ray, or ultrasound imaging that suspicious circumstances exist, a biopsy is performed. The biopsy will help determine whether the cells are cancerous, the type of cancer, and what treatment should be used to treat the cancer. Biopsy may be done by an open or percutaneous technique. Open biopsy, which is an invasive surgical procedure using a scalpel and involving direct vision of the target area, removes the entire mass (excisional biopsy) or a part of the mass (incisional biopsy). Percutaneous biopsy, on the other hand, is usually done with a needle-like instrument through a relatively small incision, blindly or with the aid of an imaging device, and may be either a fine needle aspiration (FNA) or a core biopsy. In FNA biopsy, individual cells or clusters of cells are obtained for cytologic examination and may be prepared such as in a Papanicolaou smear. In core biopsy, as the term suggests, a core or fragment of tissue is obtained for histologic examination which may be done via a frozen section or paraffin section. One important area where biopsies are performed is the diagnosis of breast tumors.

Traditionally, the biopsy technique for breast tumors involves placing a biopsy device multiple times into the breast and taking several samples of tissue from a mass or tumor which is suspected of being cancerous. Several samples are required to be sure that some tissue from the suspect mass has been captured, and enough tissue has been sampled to ensure that, if disperse cancer cells exist in the suspect mass some of those cancer cells will be captured in the samples. Each time the device is placed the physician must locate and direct the device with ultrasound imaging into the correct position near the suspect mass. Some breast tumors and lesions are very well defined, hard spherical masses which grow within the soft, compliant breast tissue. It is difficult to force a needle into these lesions because they are resistant to puncture and fairly mobile. Forcing the biopsy needle into the lesion is like trying to spear an apple floating in water.

Vacuum assisted biopsy system proposed by Biopsys involves sucking a breast lesion into a cannula and shearing off the captured edge of the lesion to obtain a biopsy sample. The device uses a vacuum to collect tissue into the side of an open tubular device, and then uses a rotating corer to cut the tissue collected. The rotating corer is slidable within the tubular section and can be pulled back to remove the tissue collected in the rotating corer. An additional stylet inside the rotating corer can be used to push the tissue out of the corer. The device can be rotated on its axis to remove a sample, 360 degrees around the central placement of the device. Typically, physicians sample six to eight cores. One advantage of this device is that the physician does not have to remove the device for additional biopsy samples. However, the tumor itself must be re-engaged after every coring operation, which entails substantial effort in relocation and confirmation that the target suspect mass has been engaged by the side aperture. Tumors may be too tough to yield to the suction and deform as necessary to enter the side opening of the cannula. Doctors also currently use the device to take a circular sequence of cores by rotating the device about its long axis or by sideways movement of the suction head to take a line of cores.

After biopsy and analysis, the tumor must be treated with a separate device, as Biopsys teaches that their coring device should not be used for resection. Indeed, the device is not designed to perform resection with assurance that complete resection of a suspect mass has been accomplished. Mechanical cutting and disruption of the tissue structure and cancer cell dispersion (that is, tearing of the tissue around the cancer and movement of the cancer cells amongst normal tissue) will result in unintentional delivery of cancer cells into healthy tissue adjacent the lesion.

In addition to the obstacle of re-engaging tumors with current vacuum assisted biopsy systems, these current biopsy systems pose additional obstacles when used with diagnostic equipment such as Magnetic Resonance Imaging Equipment (MRI). Current vacuum assisted biopsy systems contain many components that are subject to interference with MRI fields. This interference prevents diagnostics procedures from being performed while the biopsy systems are near a patient. Interference makes it difficult for the medical professional to locate the tumor and verify the location of the biopsy system before and during a biopsy. Because of the many drawbacks found in current vacuum assisted biopsy systems, there remains a need for improvements in biopsy systems.

### SUMMARY

The device described below provides for diagnosis of tumors within the breast. The device includes an adhesion probe with structures that permit the surgeon to secure a suspect mass or tumor within the breast during the biopsy procedure. The probe is provided with a rigid tube and a sharp distal tip. To secure the tumor to the probe, the surgeon pierces the tumor with the distal rod. Tubing extending within the rigid tube directs coolant to the distal tip to cool the tip resulting in the tumor adhering to the cooled probe.

The device also includes a coring apparatus with structures that permit the surgeon to core a sample of the tumor during the biopsy procedure. The coring apparatus is provided with an outer cutting cannula that advances through a tumor to core a sample of the tumor. The coring apparatus is adapted for use with the probe. The adhesion probe is disposed within the cannula with the distal tip of the probe extending beyond the distal tip of the cannula. The device is inserted into the body until the adhesion probe pierces the tumor. Coolant is directed to the distal tip of the probe to lightly cool the distal tip and the tumor. The lightly cooled distal tip adheres to the tumor cells immediately proximate the distal tip. Once the tumor is secured to the probe, the coring apparatus is actuated to excise tumor tissue surrounding the distal tip. The coring apparatus comprises a cutting cannula and means for rotating and translating the cutting cannula. After coring is complete, the device is removed from the body and the cutting cannula is retracted to release the excised tissue. This method of biopsy prevents destruction of the tumor cells and reduces seeding (the dispersion of tumor cells to healthy cell areas).

Small canisters of CO₂₁ (carbon dioxide) or N₂O (nitrous oxide), sometimes referred to as whippets, provide the coolant to the device. These small canisters eliminate the need for hoses remotely connected to large coolant canisters and allow the surgeon to freely operate during a procedure without the possibility of severing or tangling coolant supply tubes. The use of liquid CO₂ facilitates rapid yet moderate freezing of the target tissue lesion proximate the adhesion probe. The larger heat capacity of the liquid cryogen, vis-à-vis gaseous cryogen such as Argon gas, allows for further miniaturization of the reservoir and cooling probe components, with an overall gain of cooling efficiency and faster cooling operation. The liquid CO₂ is also used to drive the rotation and longitudinal translation of the biopsy coring apparatus. The system is controlled with various electromechanical interlocks and a microchip programmed to operate the system in response to operator input and various predetermined parameters.

The adhesion probe and coring apparatus are provided in a releasable coring module that can readily be inserted into and removed from a reusable control housing. The releasable coring apparatus is easily releasable since it can be operably coupled and uncoupled from the control housing without the need of additional tooling. The control housing and the releasable coring apparatus are easily carried and manipulated by the hand of a user. The coring apparatus may be manufactured from MRI compatible materials. Determining the MRI compatibility of materials requires evaluating materials for movement, artifact creation, heating, electric current induction and operation during exposure to MRI fields. MRI compatible materials includes materials that do not unpredictably move as a result of magnetic attraction or have adverse side effects such as heating up or leaking when exposed to MRI fields. These materials do not include ferromagnetic materials. Furthermore, MRI compatible materials can also include materials that create little or no image artifacts during an MRI procedure. A sufficient amount of MRI compatibility is required diagnostic procedures to be performed safely and successfully.

### BRIEF DESCRIPTION OF THE DRAWINGS

FIG. 1 is an isometric view of the biopsy instrument.

FIG. 2 illustrates the user interface of the biopsy instrument illustrated in FIG. 1.

FIG. 3 is an isometric view of internal components of the rotational core biopsy instrument in FIG. 1.

FIG. 4 illustrates a side view of the internal components of the control housing of the biopsy instrument.

FIG. 5 illustrates an elevated top view of the internal components of the rotational core biopsy instrument.

FIG. 6 shows a side view of the releasable coring module in the rotational core biopsy instrument.

FIG. 7 shows the distal tip of the cutting cannula of the rotational core biopsy instrument.

FIG. 8 shows a cross-sectional view of the cutting cannula and the adhesion probe in the rotational core biopsy instrument.

FIG. 9 illustrates the biopsy instrument with the cutting cannula in the retracted position.

FIG. 10 illustrates the translating mechanism in the advanced position.

FIG. 11 illustrates a detailed view of the distal closure head found in the coring actuator.

FIG. 12 illustrates a detailed view of the proximal closure head found in the coring actuator.

FIG. 12A illustrates the annular space between the first O-ring second O-Ring in the proximal closure head.

FIGS. 13A, 13B, 13C and 13D are schematic diagrams of the valve assembly and associated tubing for operating the biopsy instrument of FIG. 1.

### DETAILED DESCRIPTION OF THE INVENTIONS

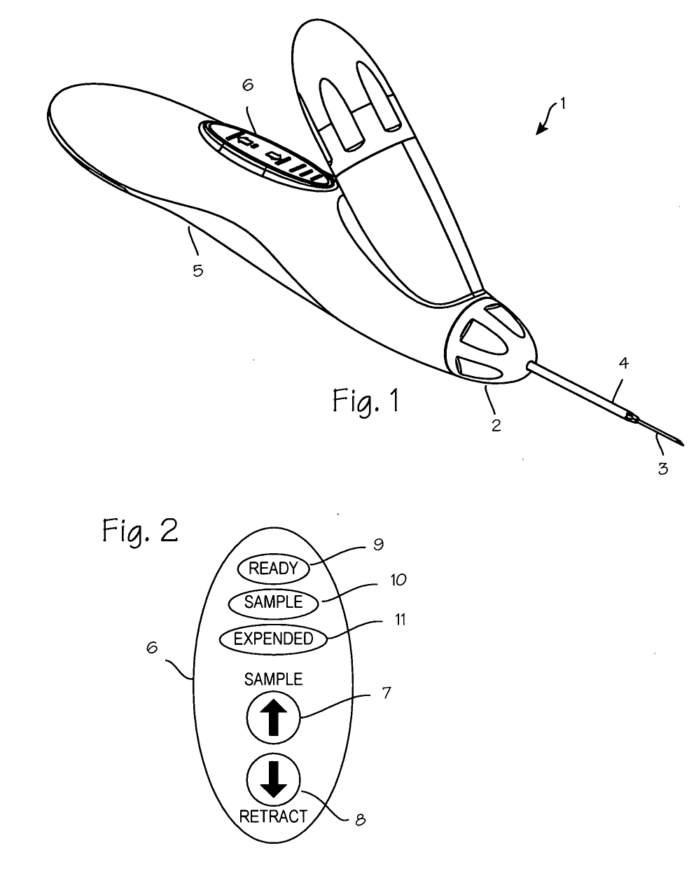

FIG. 1 illustrates a biopsy instrument 1 which comprises a releasable coring module 2 having an adhesion probe 3 and a cutting cannula 4 and a control housing 5 having a chamber sized and dimensioned to accommodate the releasable coring module. The control housing is further sized and dimensioned to form a convenient handle and to house other components of the biopsy instrument. The housing also comprises a button interface 6, detailed in FIG. 2, which allows the user to control the device and which reports to the user the state of the device. The button interface comprises a sample button 7 which may be depressed by the user to initiate sampling operation of the device, a retract button 8 which may be depressed by the operator to initiate retraction of the cutting cannula after sampling, a ready light 9 which is operable by the device controller to indicate to the operator that the device is ready for use, a sample light 10 which is operable by the control system to indicate that the device is operating to core a biopsy sample from a patient, and an expended light 11 to indicate when the CO₂ liquid has been expended. Membrane switches or other input devices may be used as input buttons. Visual, audible, or tactile indicators capable of providing several distinct signals to the user may also be used in lieu of indicator lights.

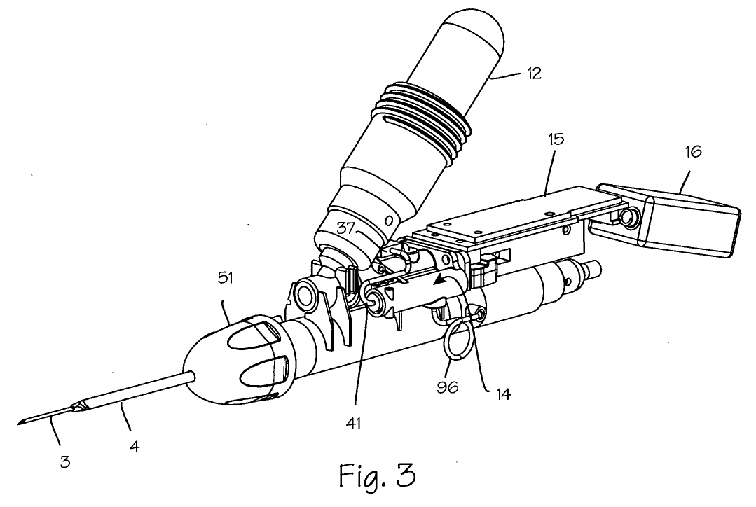

FIG. 3 is an isometric view of internal components of the of the biopsy instrument illustrated in FIG. 1. The adhesion probe 3 and cutting cannula 4 contained in the releasable coring module are operably connected to the various components of the control housing as illustrated in FIG. 3. Coolant is supplied to the adhesion probe from the small portable liquid CO₂ canisters or whippets 12. Canisters of N₂0 (nitrous oxide), ethane, propane, methane or tetrafluoromethane (R14) may also be used. Because the device is designed to use the liquid cryogen, rather than the gaseous cryogen, within the canister, the canister is held in fixed relationship to the biopsy instrument, with the outlet pointing downward, establishing an up-and-down orientation for the device. In use, the canister is disposed in an inclined position when the adhesion probe is horizontally oriented.

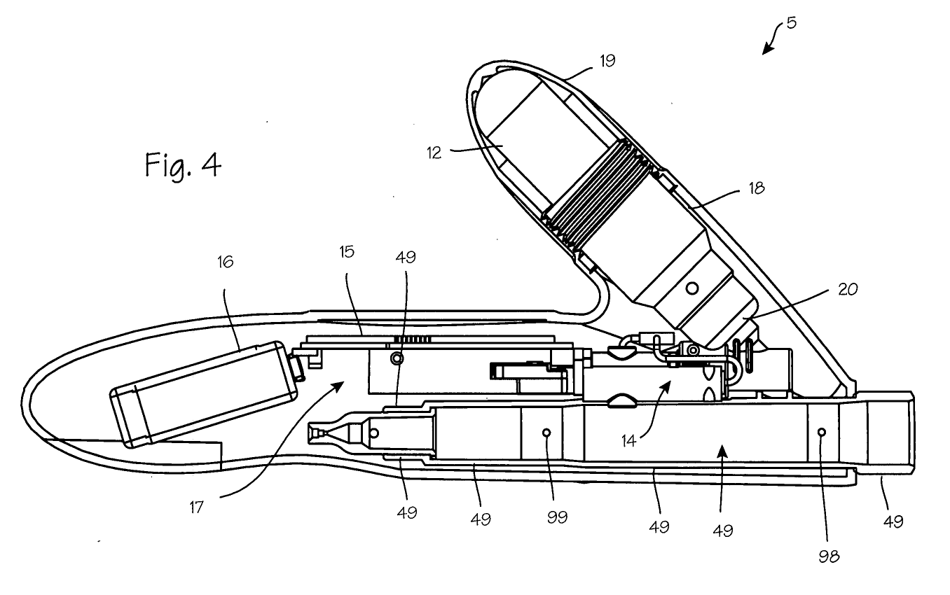

FIG. 4 illustrates a side view of the internal components of the control housing 5. The control housing contains a CO₂ canister 12, a valve block 14, a computerized control system 15, a battery 16, a motor and gear box 17 and a plurality of fluid pathways. The CO₂ canister 12 shown is disposed within a canister housing 18 and is held in place by a screw-cap 19. The outlet of the CO₂ canister is forced into a pierce pin connector 20 upon closure of the screw-cap 19, establishing a fluid pathway from the canister to the valve block 14. A chamber 49 sized and dimensioned to receive the releasable coring module is also shown. A filter may be disposed in the exhaust fluid pathway to prevent cryogen from escaping (small bits of solid CO₂, or droplets of liquid N₂O, may be ejected from the proximal end of the adhesion probe). The computerized control system is provided on a printed circuit board. The control system 15 is powered by a 9 volt battery 16 or other suitable power source. The battery can be removed if needed to facilitate disposal.

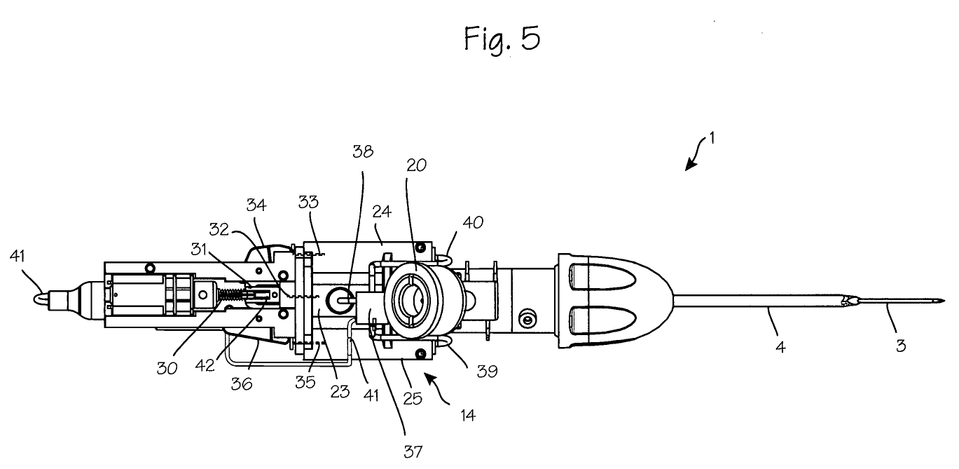

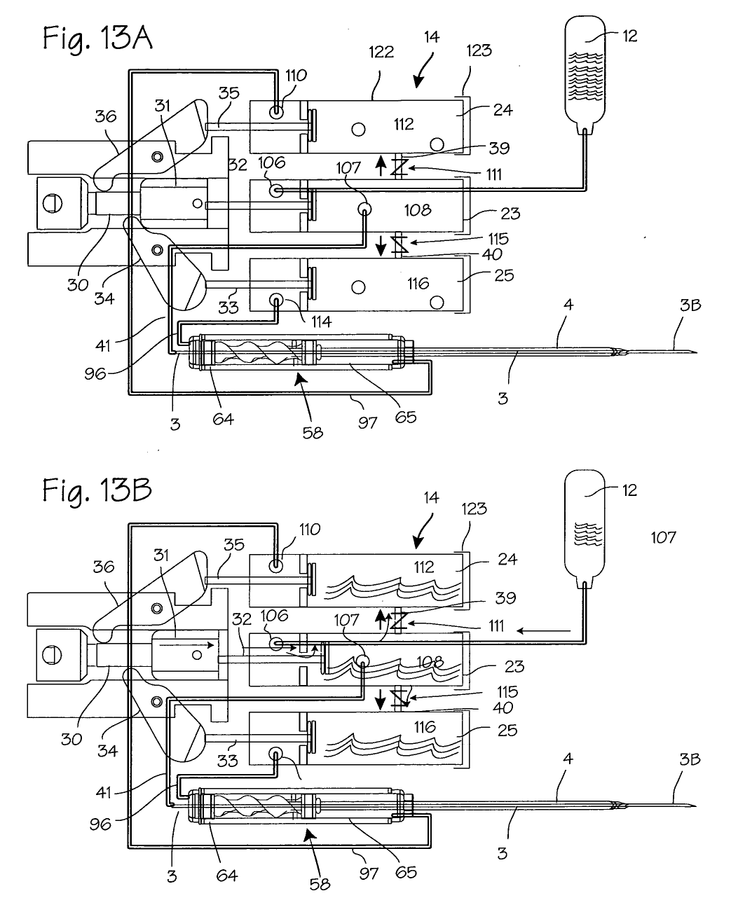

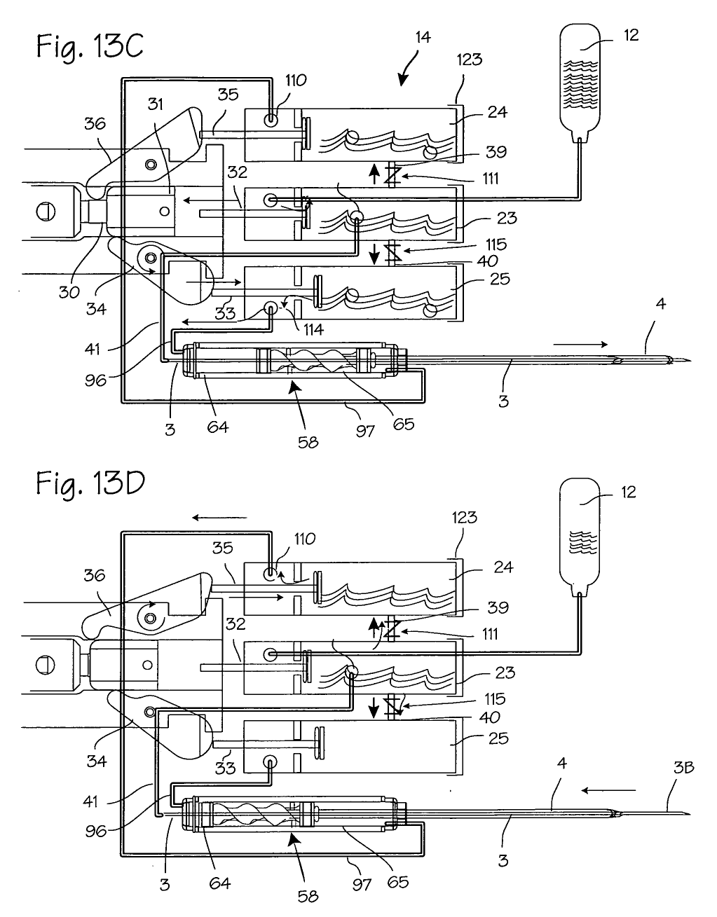

FIG. 5 shows a top view of the internal components of the biopsy device 1, in which the valve block 14 and gear motor 17 are more clearly visible. The main valve 23, advance valve 24 and retract valve 25 are connected through various fluid pathways or tubes which direct fluid flow to the various components, as described in relation to FIGS. 13A through 13D.

The motor and gearbox 17 is shown at the proximal end of the device, proximal of the valve block 14. The motor is operably connected to the various valve stems (see FIGS. 13A through 13D) through a motor gearbox, jackscrew 30 and jackscrew nut 31. The jackscrew nut operates directly on the main valve stem 32, and operates the advance valve stem 33 through cam 34, and operates on the retract valve stem 35 through cam 36. Proximal movement of the jackscrew nut results in operation of cam 34 to impinge on advance valve stem to open the advance valve and direct flow of high pressure liquid cryogen to the advance side of the cutter actuator piston, and further proximal movement of the jack screw nut results in rotation of cam 36 to impinge on retract valve stem, pushing the retract valve stem 35 into the retract valve to open the valve and direct flow to the retract side of the cutter actuator piston.

A manifold 37 is used to distribute liquid cryogen from the main valve to the various points in the system. Main valve outlet tube 38 provides a fluid pathway from the main valve to the manifold, and the fluid is then distributed to the retract valve through retract valve supply tube 39, to the advance valve through the advance valve supply tube 40 and to the adhesion probe through the adhesion probe supply tube 41. The small wiper contact 42 on the drive nut interacts with a corresponding trace on the printed circuit board disposed above the drive nut as shown in FIG. 4. The contact 42 is in predetermined position relative to the valve stem such that the wiper/trace combination may act as a limit switch to provide feedback to the computer control system as to the position of the drive nut and main valve stem. This trace and/or additional traces on the circuit board can be used as described below to provide feedback for control of the motor and drive nut.

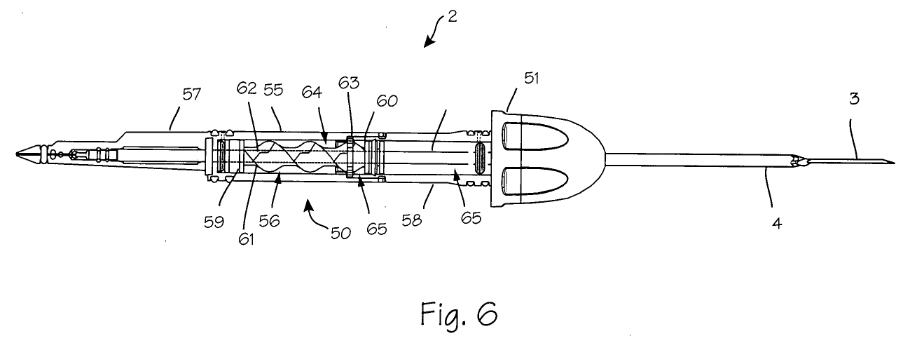

FIG. 6 shows a side view of the releasable coring module in the biopsy instrument. The biopsy instrument includes a releasable coring module having a coring actuator that serves as both a translating mechanism and a rotating mechanism for the cannula. As illustrated in FIG. 6, the translating mechanism and rotating mechanism are provided in a combined translating/coring mechanism which performs the rotating operation in conjunction with the longitudinal translation of the cannula and facilitates the coring of tissue. The releasable coring module is manufactured from materials that are MR safe. These materials include polymers, elastomers, composites, brass, aluminum, and ceramics. Because the releasable coring module may be manufactured from materials that do not move or heat up during an MRI, the releasable coring module may be placed in or near a patient during diagnostic procedures such as an MRI. This allows a surgeon to verify the location of the adhesion probe prior to a biopsy by inserting the needle and cannula into a patient prior to performing MRI and verifying placement using the MRI system. The biopsy may be accomplished immediately after verification by placing the housing over the coring module and coupling the module to the housing.

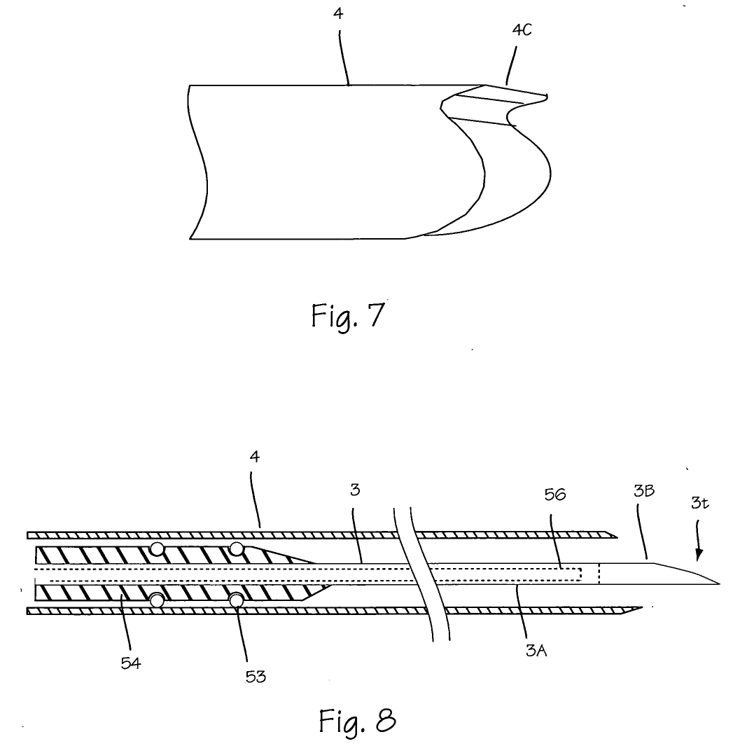

The releasable coring module 2 is adapted for releasable coupling to the control housing 5, by which we mean the releasable coring module 2 can be can be operably attached and detached from the control housing 5 easily without tooling. The releasable coring module 2 is adapted to be disposed within the chamber 49 shown in FIG. 4. The releasable coring module 2 comprises an adhesion probe 3, cutting cannula 4, a coring actuator 50, and a releasable attachable fitting 51. The fitting may be a snap fitting, slip fitting, flanged fitting, threaded fitting, grooved fitting or other apparatus suitable for releasably coupling the coring module to the housing. By releasable we mean having the ability to operably couple and uncouple the releasable coring module 2 to the control housing 5 without the use of special tooling or great effort by the user. Alternatively, the fitting may also be rotatably coupled to the housing while threads may be disposed on the coring module. When the releasable coring module is disposed within the housing, the adhesion probe 3, cutting cannula 4, and coring actuator 50 are operably connected to the various components in the control housing 5. FIG. 7 illustrates the distal tip of the cutting cannula while FIG. 8 illustrates a cross-sectional view of the adhesion probe and cutting cannula.

As illustrated most clearly in the side view of FIG. 6, the coring actuator is symmetric along its longitudinal axis. The coring actuator includes a cylinder 57 having a cylinder or piston chamber 58, a proximal closure head 59 and a distal closure head 60, a proximal piston 61 and a distal piston 62, a actuator rod 53 and lead screw or jackscrew 52 disposed between the proximal 61 and distal 62 pistons. The lead screw 52 is bounded on both sides by the pistons 61 and 62. The cylinder 58 is sized and dimensioned to fit within the chamber 49 of the control housing. The pistons 61 and 62 are disposed within the piston cylinder 58, such that the pistons, although tightly fitting within the cylinder, are capable of translating longitudinally along the cylinder. The pistons may rotate relative to the cylinder.

The pistons in FIG. 6 are attached to the cutting cannula and may be longitudinally fixed to the cannula 4 through the actuator 50. The actuator rod 53 may be integrally formed with the cannula, and may, as illustrated, be formed of the proximal extent of the cutting cannula. The pistons may be rotatably fixed to the cannula, but the cannula may also be longitudinally fixed to the pistons while remaining freely rotatable relative to the pistons. The pistons may be manufactured from a polymer or elastomer. The translation of the pistons distally and proximally through the cylinder is translated to the cannula either by the pistons, by the jackscrew between the pistons or both. The actuator rod or proximal extent of the cannula may extend proximally through a bore in the distal piston, through the lead screw, and the proximal piston or it may terminate proximally at the distal piston, or any point in between, so long as it is longitudinally fixed to the pistons and rotationally fixed to the jackscrew. In the embodiment shown, the adhesion probe and cannula are coaxially disposed within the pistons, lead screw, actuator rod and cylinder.

The coring actuator in FIG. 6 comprises a lead screw 52 (which also serves as a piston or actuator rod) and a lead screw nut 63 separating the cylinder into an advance section 64 and retract section 65. The lead screw 52 is a hollow tube having a lumen extending therethrough with screw threads extending from the outside surface of the tube. The lead screw 52 is rotationally fixed to the cutting cannula and/or the pistons, such that translation of the pistons and/or lead screw translates the cutting cannula. The lead screw nut 63 is adapted to receive the lead screw. The lead screw 52 is screwed into and through the lead screw nut. The cannula 4 is rotationally fixed to the lead screw, either directly or indirectly. When the pistons 61 and 62 are translated, the lead screw 52 moves longitudinally under the operation of the pistons and rotates via translation of the lead screw 52 through the lead screw nut 63. Because the cannula is rotationally fixed to the lead screw, the rotation of the lead screw is translated to the cannula. The lead screw can have a pitch of 1 inch per revolution, such that for every inch of translation, the lead screw rotates one revolution.

As seen in FIG. 7, the cutting edge is provided in the form of a scalloped bevel, formed by multi-axis machining. Thus, the cutting edge 4c has several distally extending, longitudinally rounded protrusions which are provided with a sharp longitudinally oriented bevel, with the cutting edge toward the inner wall of the cannula, and the bevel extending proximally toward the outer wall, and the circumference of the bevel following a scalloped or sinusoidal curve relative to the longitude of the cannula. A beveled tip with three such longitudinally rounded protrusions works well on breast tissue.

FIG. 8 illustrates details of the cutting cannula and adhesion probe. The adhesion probe 3 and cutting cannula 4 of the prior figures are shown in cross section. A ferrule 54 is fitted coaxially over the adhesion probe, between the adhesion probe and the cutting cannula. The ferrule is fixed to the adhesion probe, and has an outer diameter closely matching the inner diameter of the cutting cannula, and is used to provide the proximal segment of the adhesion probe with a larger outer diameter than the distal segment. A trocar-type blade or tapered cone provides the transition from the outer diameter of the ferrule to the distally extending penetrating segment 4C is formed in. One or more ring seals 55 are disposed between the ferrule and the cutting cannula, and may be secured within annular grooves within the ferrule, as shown. The ring seals serve to prevent body fluids seeping into the clearance between the adhesion probe/ferrule and the cutting cannula and freezing during operation of the device, and this eliminates any interference with cannula translation that may result.

The adhesion probe 3 shown in FIG. 8 comprises a long, slender yet rigid outer tube 3A. A short rigid penetrating segment 3B extends distally from the distal end of the rigid tube, and a coolant inlet tube 56 passes through the rigid tube, extending to the distal end of the outer rigid tube 3A, and terminating just proximal of the distal tip of the penetrating segment 3B. The adhesion probe may be manufactured from stainless steel, aluminum, bronze, polymers or other MRI compatible materials. The distal tip 3t is beveled, and the bevel face is oriented to face upward relative to the device, and thus is radially aligned with the cryogen canister, so that it is oriented superficially, toward the skin of the patient, when in use. A suitable adhesion probe is described in Spero, et al., Rotational Core Biopsy Device with Liquid Cryogen Adhesion Probe, U.S. patent application Ser. No. 10/779,520 (Feb. 12, 2004), incorporated herein by reference. The cutting cannula 4 is slidably disposed around the adhesion probe 3, longitudinally translatable relative to the adhesion probe and adapted for insertion through a small incision in the skin, and may be inserted along the with the adhesion probe or over the adhesion probe. The cutting cannula may be forced distally over the penetrating segment 3B to core any tissue which is secured to the penetrating segment from any surrounding body tissue.

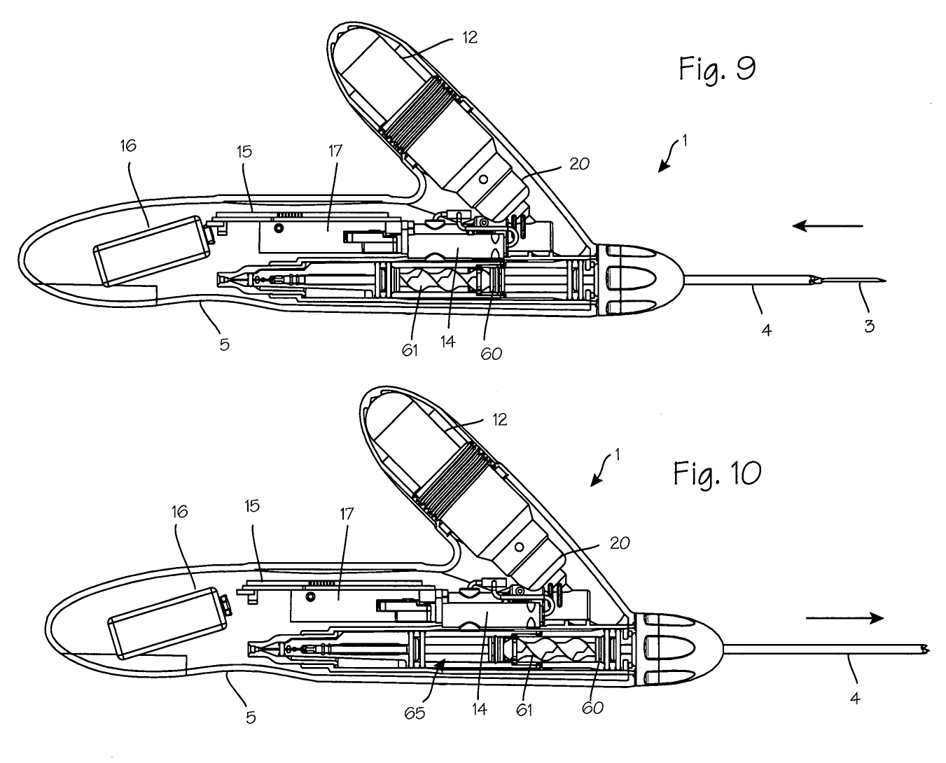

The lead screw of the actuator and the cutting cannula 4 have a retracted/proximal position and an extended/distal position. FIG. 9 illustrates the lead screw 52 the cutting cannula 4 in the retracted position, in which the cannula 4 will not be engaged with the tumor. In the retracted position, the distal piston 62 is positioned at the proximal end of the cylinder and the penetrating segment 3d of the adhesion probe 3 is exposed, extending distally from the cannula 4. FIG. 10 illustrates the lead screw in the advanced position, with the cannula translated distally over the penetrating segment 3d of the adhesion probe, where it will engage and core a tumor secured to the penetrating segment. Comparison of FIGS. 9 and 10 illustrates the cooperative interaction between the proximal and distal pistons, the lead screw 52, and the cutting cannula 4. FIGS. 9 and 10 also show the CO₂ canister 12, the valve block 14, the computerized control system 15, the battery 16, the motor and the gear box 17, all within the housing 5. The CO₂ canister 12 is shown disposed within the vertically oriented canister housing (formed integrally with the housing 5) and is held in place by the screw-cap. The outlet of the CO₂ canister is forced into the pierce pin connector 20 upon closure of the screw-cap, establishing a fluid pathway from the canister to the valve block. The CO₂ used in the probe exhausts from the proximal end of the adhesion probe. A filter is disposed in the exhaust gas pathway to prevent cryogen from escaping (small bits of solid CO₂₁ or droplets of liquid N₂O, may be ejected from the proximal end of the adhesion probe).

FIG. 11 illustrates an obstructed view of the distal closure head 60 found in the coring actuator 50 shown in FIG. 6. The distal closure head comprises a body characterized by a distal section 73 and proximal section 74. The body of the distal closure head may be manufactured from a non-ferrous alloy such as brass or a polymer. The distal section is coupled to a rotatable internally threaded fitting 51 sized and dimensioned to couple to the control housing. The proximal section of the distal closure head contains a first annular groove 75 and a second annular groove 76. A first O-ring 77 is disposed within the first annular groove and a second O-ring 78 is disposed within the second annular groove. An opening 79 to a lumen in fluid communication with the retract section 65 of the cylinder is disposed between the first and second groove.

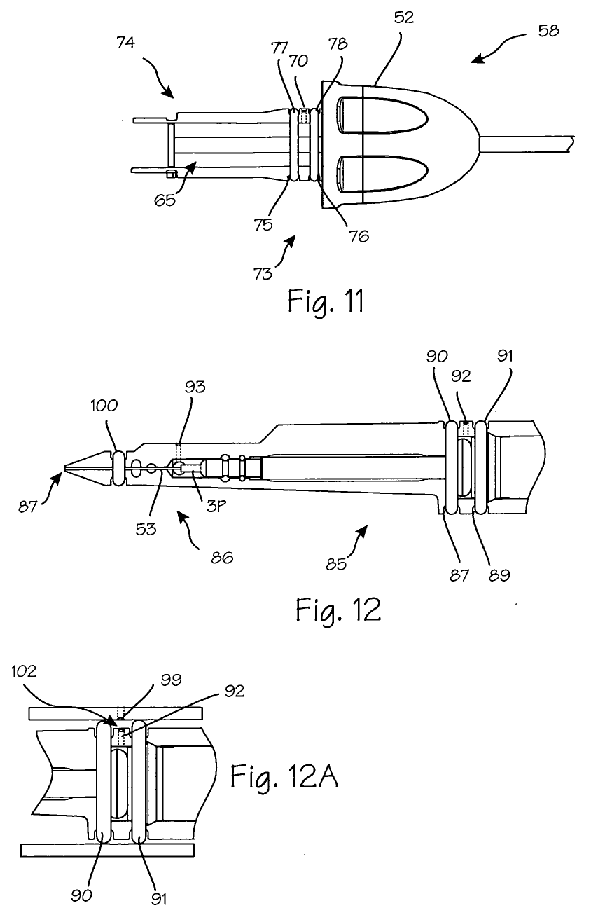

The proximal closure head 59 in the coring actuator as shown in FIG. 12, can also be manufactured from a non-ferrous alloy, polymer or other material with reduced electro-magnetic interference. The proximal closure head comprises a body characterized by a distal section 85 and proximal section 86. A coolant supply lumen 87 extends longitudinally through the proximal closure head and is in fluid communication with the coolant inlet tube 55 of the adhesion probe 3. An opening to the coolant supply lumen is disposed on the proximal tip of the proximal closure head. The distal section of the proximal closure head contains a first annular groove 88 and a second annular groove 89. A first O-ring 90 is disposed within the first annular groove and a second O-ring 91 is disposed within the second annular groove. An opening 92 to a lumen in fluid communication with the advance section of the cylinder is disposed between the first and groove. The proximal closure head also comprises a cut out or opening 93 in the proximal section allowing exhaust fluids to escape the outer rigid tube of the adhesion probe.

When the releasable coring module 2 is disposed within the control housing 5 it is removably coupled to the chamber. The threaded fitting 51 found in the releasable coring module is screwed over threads in the distal section of the control housing to seat releasable coring module 2 within the chamber. An outlet 98 for a retract cylinder supply tube 97 originating from the retract valve is located in the distal section of the chamber while outlets for an advance cylinder supply tube 96 originating from the advance valve and the adhesion probe supply tube are disposed in the proximal section of the chamber. An annular space between the first O-ring 77 and second O-Ring 78 in distal closure head allows fluid communication between the retraction supply tube 97 and the opening 79 of the lumen to a retract section 65 of the cylinder in the coring actuator. When the releasable coring module is disposed within the chamber the outlet for the retract cylinder supply tube 97 is placed in fluid communication with this annular space allowing fluid from the retract valve to flow into this space and through the opening 70 and into the retraction section of the cylinder.

Similarly, as shown in FIG. 12A, an annular space 102 between the first O-ring 90 second O-Ring 91 in the proximal closure head allows fluid communication to occur between the advance supply tube 40 and the opening of the lumen 92 to the advance section of the cylinder in the coring actuator. When the releasable coring module 2 is disposed within the chamber, the outlet 99 for the advance cylinder supply tube 40 is placed in fluid communication with this annular space 100 allowing fluid from the advance valve to flow into this space and into the opening for the lumen to the advance section of the cylinder.

While the releasable coring module is disposed within the control housing, the opening of the coolant supply lumen in the proximal closure head is placed in fluid communication with the adhesion probe supply tubing 41 through the proximal port in the chamber 49. This places the adhesion probe in fluid communication with CO₂ canister or other coolant source. O-ring 101 seals this flow path from the remained of the chamber 49.

FIGS. 13A through 13D are schematic diagrams of the valve assembly and associated tubing for operating the biopsy device of FIG. 1. The valve assembly comprises the valve block 14, which includes a main valve 23 and two associated valves 24 and 25 which have inlets aligned to the outlet of the main valve. The main valve 23 comprises a main valve stem 32, main valve outlet 106, main valve inlet 107 and main reservoir 108. The retract valve 25 comprises a retract valve stem 35, retract valve outlet 110, retract valve inlet 111 and retract reservoir 112. The advance valve 24 comprises an advance valve stem 33, advance valve outlet 114, advance valve inlet 115 and advance reservoir 116. The valves are spring loaded plunger valves which are normally closed, such that movement of the plunger into the valves opens the valves. The valves may comprise a ball which is forced against the valve seat, or a typical plunger valve with a seal positively fixed to the plunger, as shown. The inlets of both the retract valve 25 and the advance valve 24 are fitted with check valves, which may be spring biased ball check valves or any other type of check valve.

FIG. 13A shows the system in its initial condition, with all three valves closed, the cutting cannula 4 in the retracted position, and the adhesion probe 3 extending distally from the distal extent of the cutting cannula 4. The CO₂ canister 12 is filled with liquid CO₂, and is in fluid communication with the inlet of main valve 23.

During sampling operation (which is initiated when the user depresses the sample button on the input pad shown in FIG. 2), the motor 17 operates through linkages to drive the jack screw nut 31 forward, thus driving the main valve stem 32 forward, thereby opening the main valve 23, as shown in FIG. 13B. The main valve stem 32 is driven forward from a home position until the electrically conductive wiper that is mounted to the drive nut loses contact with traces on the printed circuit board (any other form of contact switch, proximity switch, encoder or sensor may be used to sense the position of the main valve stem 32 (and, thus, the state of the valve)). The motor stops in this position for a period (the dwell time) which may be predetermined or calculated by the computerized control system. Preferably, the dwell time is calculated by the control system based on the time required for the conductive wiper to traverse the trances on the printed circuit board. This dynamic calculation of the dwell time allows the computerized control system to automatically compensate for variations in the speed of the valve stem travel due to motor characteristics, friction in the system, and battery voltage. With the main valve open, liquid C0₂ flows through the main valve outlet 106 to the adhesion probe supply tubing 41 that is connected to the proximal end of the chamber 49. (The sample light 10 is flashed while cryogen is flowing to the adhesion probe to indicate to the operator that the device is operating in cooling mode. Other distinctive indications may be provided to the operator.) The proximal end of the chamber 49 is in fluid communication with the coolant inlet tube 56 in the adhesion probe through the opening to the coolant supply lumen 87 disposed on the proximal end of the proximal closure head 59. As the cryogen enters the coolant inlet tube 56 and exhaust exits the outer rigid tube 3A of the adhesion probe 3 through the exhaust lumen 93, the temperature of the penetrating segment 3B drops. While liquid is flowing to the adhesion probe, liquid is also routed to charge the advance reservoir 116 and retract reservoir 112 through the advance valve inlet 115 and the retract valve inlet 111. The size of the reservoirs are calculated to provide a set pressure inside the cylinder once all of the valves open and the liquid CO₂ is turned to vapor with an expansion ratio of 400:1 or more (compensating for end state gas temperature).

After the dwell time, the motor is reversed. As the main valve stem 32 moves backward, as shown in FIG. 13C, the main valve closes. The motor continues in reverse operation to drive the drive nut backward. At this point, the main valve is fully closed and the cooling flow to the adhesion probe ceases. The jack screw nut encounters a cam and forces the cam to pivot forward and forces advance valve stem forward to open the advance valve. This allows fluid to flow through the pressure tubing into the advance side of the piston cylinder 58 which drives the cutting cannula forward. As the cannula translates, the cannula rotates under operation of the lead screw and lead screw nut assembly illustrated in FIGS. 9 and 10. Any tissue adhered to the tip of the adhesion probe 3 when the cannula 4 is translated and rotated is cored from the surrounding lesion. The motor continues in reverse operation to draw the drive nut backward until the wiper encounters a second contact (the second contact is located on the circuit board or other fixed structure located above the drive nut) and stops. The cutting cannula is fully extended over the adhesion probe, and has excised any tissue adhered to the distal segment of the adhesion probe. The time required for this complete stick freeze/advance cycle is preferably less than 10 seconds, and is about 4 seconds using the embodiments illustrated. The control system 15 illuminates the sample light 10 on the input pad continuously after advancing the cutter, to indicate to the operator that the coring operation is complete.

Though the electromechanical valve actuators described above in relation to FIGS. 5 and 13A through 13D provide for fairly simple, compact and quick actuation of the valves in the high pressure system, other electromechanical valve actuators may be used. Each valve may be driven by a different solenoid actuator or a different motor and each actuator or motor may be operated by the control system programmed to provide the valve timing described above. Other valve actuators, including pneumatic actuators (driven by the high pressure cryogen stored in the canister), shape memory actuators (heated by the battery, as controlled by the control system), and any other valve actuating means may be used. The embodiment described above, however, is compact, sufficiently powerful to operate against the high pressures of the cryogen, and inexpensive.

During retraction (which is initiated when the user depresses the retract button 8 on the input pad shown in FIG. 2) the control system operates the motor continues to operate, in reverse, to move the drive nut backwards until a second cam is encountered by the jackscrew nut. This second cam pivots forward and opens the retract valve 25, as shown in FIG. 13D. This allows fluid to flow through the retract cylinder supply tubing 97 into the retract side 65 of the piston cylinder, which in turn retracts the cutting cannula. The tissue excised from the body is then exposed, and is readily removed from the distal segment of the adhesion probe.

The advance side 64 of the piston cylinder 58 must be evacuated prior to application of high pressure fluid to the retract side, to prevent hydraulic/pneumatic binding of the piston. The advance side 64 of the piston cylinder may be vented in any convenient manner. In the device illustrated in the Figures, the valve bodies comprise cylinders 122 with end caps 123. The threading of end cap on the advance valve is machined so that it is slightly loose (or gas valve threads are used, and the cap is not completely seated) and allows slight leakage of the cryogen from the valve body reservoir. Thus, after the bulk of the cryogen is exhausted into the piston cylinder, the piston cylinder, the advance side exhausts through the end cap. The retract cylinder is vented in the same manner. Vented may be accomplished with small apertures in the end caps or valve bodies in similar fashion.

The amount of time in which coolant is flowing depends on desired temperature of adhesion probe. Final temperature of about −1° to −20° C. is desired for biopsy, while a final temperature below −20° C. is desired for cryo-preservation. Alternatively, a thermocouple may be embedded in the adhesion probe so that the device may be temperature controlled rather than time controlled. This will compensate for differences in device or tissue thermal loading, or the difference between the first shot of liquid CO₂ and the last as the device cools down, and for variations in the speed of the valve stem travel which may result from variations in the battery. For a standard biopsy with a fully charged battery, the dwell time after the main valve is fully open is about 0.5 to 2.0 seconds. The valve is open, then, for about 5 seconds, which includes the dwell time and the time in which the valve stem is moving (and the valve is open). CO₂ flow of 0.05 and 1.25 grams per 5 second cycle (0.01 to 0.25 grams per second) provides adequate cooling for biopsy, which requires cooling sufficient to adhere the probe to the tissue, and preferable does not result in extensive freezing. This flow is appropriate in embodiments in which the adhesion probe outer tube has an outer diameter of 0.0.43 inches and an inner diameter of 0.029 inches (a 19 gauge hypo tube), and the adhesion probe inner tube has an outer diameter of 0.020 inches and an inner diameter of 0.007 inches (28 gauge). The flow rate may be adjusted as necessary with different constructions of the device.

After moving the jackscrew nut back a set distance, the motor is stopped and then driven forward until the jack screw nut is driven to its home position. The control system checks the battery voltage and verifies that the number of cycles used is within the capacity of the CO₂ canister. Conveniently sized canisters hold enough liquid CO₂ to supply the system for about 7 coring operations. Twelve to sixteen grams of liquid are sufficient in a canister filled to 75% density. If there are any cycles left, the ready light 9 illuminates. If not, the expended light 11 illuminates and the system is software disabled. The system will not operate if it has already counted 7 operating cycles (this limit is somewhat arbitrary, chosen to provide ample cycles for a single patient use, and it may be adjusted as manufacturers and doctors gain experience with the device).

In use, the user screws down the screw cap. This drives the CO₂ canister 12 down into the pierce pin connector. When the canister is fully seated, an electrical connection is completed which “wakes up” the control system on the printed circuit board. A self-check program executes and exercises the gear motor (shown in FIG. 6) to establish a home position. The time that it takes to move the main valve stem from point to point is also measured and the valve cycle time is altered based on the measured speed to achieve a desired cryogen flow cycle time. An exemplary calculation would be:
valve cycle time=(desired cryogen flow cycle time)+(valve stem travel time to initiate flow);
where
desired cryogen flow cycle time=valve stem travel time after valve opening+dwell time.

In each case, the valve stem travel time is calculated by dividing the distance the valve must travel (which depends on the construction of the device) by the measure speed of the valve stem (which corresponds to the speed of the drive nut). The speed of the drive nut is determined by measuring the time required to travel past the trace, or to move from one trace to another trace, given that the trace(s) are fixed relative to the drive nut wiper and the length of the trace (or the distance between the traces) is known.

After a successful self-check, the ready light 9 on the button interface 6 illuminates. The user, typically a surgeon or radiologist, inserts the distal tip of the adhesion probe into a tumor or other suspect mass within the body of a patient. When the user is satisfied with the position of the adhesion probe, the user depresses the sample button on the input pad, and the system initiations the cooling and coring operation described above in relation to FIGS. 13C through 13A. After the coring operation is complete, the control system operates the sample light continuously to indicate to the operator that the sample has been cored from the patient. The user than removes the probe from the patient, and depresses the retract button on the input pad. In response, the control system initiates the retraction operation described above in relation to FIG. 13D. The cored tissue sample may then be removed from the distal tip, and, if the user desires to take more samples, the adhesion probe can be re-inserted into the body.

The system is provided with safety features to prevent over-pressurization, initiation of sampling with a partially discharged device, etc. The average pressure inside the CO₂ canister at room temperature is 850 psi. Extreme ambient heating may result in canister pressure of 3 kpsi. The burst pressure of the canister is 10 kpsi, but there is no need to construct the entire probe to withstand such high pressure. Thus, a burst disk may be placed in line with the main valve so that it will vent when the pressure is higher than 3 kpsi. Any other suitable pressure relief means may be used. In the event the probe, after having a canister installed, is set aside for an inordinately long time, the canister may self discharge, so that it no longer hold enough gas for a full compliment of sampling procedures, or doctors may inadvertently attempt to use a device on a patient after it has already been used on another patient. Thus, the control system is programmed to exhaust the probe after a predetermined time period, such as by driving the drive nut forward to vent out any remaining gas. The chance of initiating sampling with a partially charged device that may have been used with another patient is minimized.

When the biopsy instrument 1 is in use, the adhesion probe 3 in the releasable coring module 2 is inserted into a patient and manipulated into a suspect lesion. The patient and the releasable coring module 2 can then be placed into the imaging field of an MRI or other diagnostic equipment. Once in the imaging field, the patient is imaged and the location of the probe 3 with respect to the lesion can be confirmed. The patient can then be removed from the imaging field and the housing can be placed over the releasable coring module and secured to the apparatus using the fitting 51. A biopsy can then be performed using the assembled biopsy instrument 1. This process may be repeated as necessary.

While the preferred embodiments of the methods have been described in reference to the environment in which they were developed, they are merely illustrative of the principles of the inventions. Other embodiments and configurations may be devised without departing from the spirit of the inventions and the scope of the appended claims.

We claim:

1. A device comprising:
a control housing comprising a control system, a battery, a canister of liquefied gas and means for selectively supplying liquefied gas from the canister to an adhesion probe and a fluid actuator; and
a releasable coring module releasably coupled to the control housing and in fluid communication with the canister when coupled to the control housing, said coring apparatus comprising:
an cryogenic adhesion probe, said probe adapted for insertion into a mass of tissue;
a cutting cannula disposed about the adhesion probe; and
a fluid actuator coupled to said cutting cannula wherein the fluid actuator is adapted to translate the cutting cannula longitudinally and rotationally.

2. The device of claim 1 wherein the releasable coring module comprises MRI compatible materials.

3. The device of claim 1 wherein the control housing and the releasable coring module comprise MRI compatible materials.

4. A system for securing a mass within the breast of a human patient, said system comprising:
a releasable coring module comprising:
a cryogenic adhesion probe comprising a tube adapted for insertion into the body of the patient, said tube having a proximal end, a distal end, a proximal segment, and a distal segment, said proximal segment having a larger outer diameter than the distal segment; said distal segment having a penetrating element adapted for piercing the mass;
a cutting cannula disposed about the tube, said cutting cannula characterized by a proximal end and a distal end, said cutting cannula having an inner diameter larger than outer diameter of the distal segment of the adhesion probe;
disposed within a cylinder, a first chamber on one side of the first piston and a second chamber on the other side of the first piston, said first piston being longitudinally fixed to the cutting cannula; and
a control housing adapted to receive the releasable coring module, said control housing comprising a control system, a canister of liquefied gas and means for selectively supplying liquefied gas from the canister to the adhesion probe, the first chamber and the second chamber.

5. The system of claim 4 wherein the releasable coring module comprises MRI compatible materials.

6. The system of claim 4 wherein the control housing and the releasable coring module comprise MRI compatible materials.

7. A device comprising:
a housing comprising a battery, a control system in electrical communication with said battery, a motor, a cryogen fluid source and means for selectively supplying the cryogen fluid source to a coring apparatus;
wherein the coring apparatus is releasably disposed within the housing and comprises an adhesion probe, a cutting cannula disposed about the adhesion probe, and a fluid actuator coupled to the cutting cannula adapted to translate the cutting cannula longitudinally and rotationally.

8. The device of claim 7 wherein the coring apparatus comprises MRI compatible materials.

9. The device of claim 7 wherein the housing and the coring apparatus comprise MRI compatible materials.

10. A system for securing a mass within a human patient, said system comprising:
a portable control housing comprising control system, a power source in electrical communication with said control system, a fluid source, a chamber in fluid communication with the fluid source adapted to received a coring apparatus;
wherein the coring apparatus comprises an adhesion probe in fluid communication with the fluid source when the coring apparatus is disposed within the distribution chamber, a cutting cannula disposed about the adhesion probe and adapted to rotate while translating longitudinally and an actuator coupled to the cutting cannula having an advance section and a retract section, said advance section and a retract section in fluid communication with the fluid source when the coring apparatus is disposed within the distribution chamber.

11. The system of claim 10 wherein the coring apparatus comprises MRI compatible materials.

12. The system of claim 10 wherein the portable control housing and the coring apparatus comprise MRI compatible materials.
